(19) United States
(12) Patent Application Publication
(10) Pub. No.: US 2021/0370048 A1
(43) Pub. Date: Dec. 2, 2021

(54) TRANSCUTANEOUS ELECTRICAL NERVE STIMULATION DEVICE

(71) Applicant: RELEGS B.V., Noordwijk (NL)

(72) Inventors: Erik VAN DER HEIDE, Abcoude (NL); Casper VAN WAVEREN HOGERVORST, Abcoude (NL); José A CARRASCO, Alicante (ES); Francisco GARCIA DE QUIRÓS, El Campello (ES)

(73) Assignee: RELEGS B.V., Noordwijk (NL)

(21) Appl. No.: 17/309,077

(22) PCT Filed: Oct. 25, 2019
(86) PCT No.: PCT/EP2019/079218

§ 371 Date: Apr. 21, 2021

(30) Foreign Application Priority Data
Oct. 26, 2018 (NL) 2021889

(51) Int. Cl. A61N 1/04; A61N 1/36

(52) U.S. Cl. CPC A61N 1/0456; A61N 1/36034; A61N 1/36021

(57) ABSTRACT

A Transcutaneous Electrical Nerve Stimulation, TENS, device has a pair of electrodes for attachment to the skin of a user. It is determined that at least one of the electrodes is peeling, by applying constant voltage pulses to the pair of electrodes; detecting a peak current at the start of the pulse, and detecting a plateau current at the end of the pulse; comparing peak currents from a plurality of pulses; comparing plateau pulses from the plurality of pulses; and determining that at least one of the electrodes is peeling if the peak currents from the plurality of pulses decline over time and if the plateau pulses from the plurality of pulses decline over time.

### TECHNICAL FIELD

This invention relates to Transcutaneous Electrical Nerve Stimulation (TENS), which is the use of a mild electrical current to stimulate the nerves of a human or animal patient, for therapeutic or other purposes.

### BACKGROUND

TENS may be used for the alleviation of restless legs syndrome (RLS), periodic limb movement disorder (PLMD), central sensitivity syndromes, and pain, including chronic pain.

However, there is a need to improve existing TENS devices, in order to improve their usability, in particular for RLS or other patients.

### SUMMARY

According to a first aspect of the invention, there is provided a method of operation of a Transcutaneous Electrical Nerve Stimulation, TENS, device, the method comprising:
applying constant voltage pulses to a pair of electrodes attached to the skin of a user;
during a plurality of said pulses, detecting a peak current at the start of the pulse, and detecting a plateau current at the end of the pulse;
comparing peak currents from the plurality of pulses;
comparing plateau pulses from the plurality of pulses; and
determining that at least one of the electrodes is peeling if the peak currents from the plurality of pulses decline over time and if the plateau pulses from the plurality of pulses decline over time.

According to a second aspect of the invention, there is provided a Transcutaneous Electrical Nerve Stimulation, TENS, device, the device comprising:
a pair of electrodes, for attachment to the skin of a user; and

a processor, wherein the processor is programmed to perform a method in accordance with the first aspect.

According to a third aspect of the invention, there is provided a method of operation of a Transcutaneous Electrical Nerve Stimulation, TENS, device, the method comprising:
while the device is in operation, generating pulses to be applied to a pair of electrodes;
using an accelerometer to detect taps on the device;
in response to a first predetermined pattern of user-generated taps, altering an intensity of said pulses in accordance with a predetermined sequence of intensities; and
in response to a second predetermined pattern of user-generated taps, deactivating the device.
According to a fourth aspect of the invention, there is provided a Transcutaneous Electrical Nerve Stimulation, TENS, device, the device comprising:
pulse generation circuitry, for generating pulses to be applied to a pair of electrodes;
an accelerometer, for detecting taps on the device; and

a processor, wherein the processor is programmed to perform a method in accordance with the third aspect.

According to a fifth aspect of the invention, there is provided a Transcutaneous Electrical Nerve Stimulation, TENS, device, comprising a stimulation unit a pair of electrodes, and a storage case,
wherein the stimulation unit comprises:
a battery;
a charging controller; and
an output, for supplying electrical power from the battery to the pair of electrodes; and
wherein the storage case comprises:
a connector for connection to an external power source;
means for supplying electrical power to the battery under control of the charging controller, when the battery is located in a storage position in the storage case.

The TENS device may further comprise a protective cover.

According to a sixth aspect of the invention, there is provided a method of detection of component failure in a Transcutaneous Electrical Nerve Stimulation, TENS, device, the method comprising:
applying a series of positive and negative voltage pulses to a pair of electrodes attached to the skin of a user;
measuring first and second currents during the positive and negative voltage pulses respectively;
determining whether at least one of the first and second currents is outside an expected range; and
if at least one of the first and second currents is outside an expected range, identifying a component failure in the TENS device.

According to a seventh aspect of the invention, there is provided a Transcutaneous Electrical Nerve Stimulation, TENS, device, the device comprising:
pulse application circuitry, for applying a series of positive and negative voltage pulses to a pair of electrodes to be attached to the skin of a user; and
a processor, wherein the processor is programmed to perform a method in accordance with the sixth aspect.

Thus, the TENS device according to the present invention takes into account the activity of RLS patients and the ease of use of the device in different postures and situations.

### BRIEF DESCRIPTION OF DRAWINGS

FIG. 1 is a schematic view of a system including a TENS device, and storage case.

FIG. 2 illustrates parts of the TENS device in more detail.

FIG. 3 is an electrical circuit diagram of electrical circuitry in the TENS device in one embodiment.

FIG. 4 is an electrical circuit diagram of electrical circuitry in the TENS device in another embodiment.

FIG. 5 illustrates a form of one positive current pulse in use of the device.

FIG. 6 is a flow chart illustrating a first method of control of the device.

FIGS. 7, 8, 9, 10, and 11 illustrate voltage and current pulses in use of the device.

FIG. 12 is a flow chart illustrating a second method of control of the device.

FIGS. 13, 14, 15, and 16 illustrate the form of the TENS device.

### DETAILED DESCRIPTION

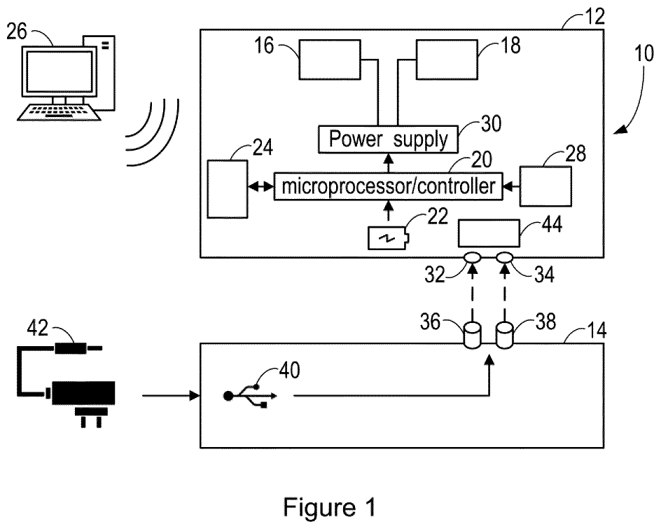

FIG. 1 is a schematic overview of a Transcutaneous Electrical Nerve Stimulation (TENS) device 10, which can be used to apply electrical currents to particular areas of the human body, for example for the alleviation of restless legs syndrome (RLS), periodic limb movement disorder (PLMD), central sensitivity syndromes, and pain, including chronic pain.

In the example device shown in FIG. 1, there is a stimulation unit 12 and a separate storage case 14.

The stimulation unit 12 is sufficiently lightweight, slim and small, that it can be worn comfortably and unseen under clothing (even tight clothing) in public, and in bed. In addition, the stimulation unit 12 is robust enough for intensive portable use and in the event of severe limb movements. Further, the stimulation unit 12 is watertight, so that safety and operations are still assured when accidentally taking a shower while wearing the device, for example.

The stimulation unit has a pair of electrodes 16, 18, which are designed so that they can be placed on a user's body, for example on an upper calf, upper leg, or lower back to provide electrical stimulation in these areas as required, depending on the user's need. The electrodes 16, 18 may for example be made of silver coated carbon. The electrodes are placed on the skin of the user within, adjacent to, or proximal to, the area of pain. The electrodes typically utilize hydrogels to create a stable low-impedance electrode-skin interface to facilitate the delivery of electrical current to the user so as to stimulate peripheral sensory nerves.

In the illustrated embodiments, the electrodes are adhesive, so that they can be placed on the user's skin and then stick to the user's skin. Specifically, the electrodes are manufactured with skin-compatible glue to stick the electrodes to the user's skin without causing allergies.

The stimulation unit 12 is based on a microprocessor/controller 20, which receives its power supply from a rechargeable battery 22, for example a Li-ion battery, which for example has an energy capacity of 370 mAh. This allows the stimulation unit 12 to provide electrical stimulation at the required level for several hours.

The stimulation unit 12 includes a short range communications module 24, for communicating with a separate device, for example a personal or laptop computer 26 or a smartphone, running a suitable control application. For example, the short range communications module 24 may be able to communicate with the separate device 26 using the Bluetooth™ communications protocol or another suitable communications protocol.

The stimulation unit 12 further includes an accelerometer 28, for detecting movement of the device, for example caused by the user tapping on the unit or by the movement of the part of the user's body to which the device is attached.

As described in more detail below, the TENS device operates by generating electrical stimulation pulses with specified characteristics. Thus, in this case, the stimulation unit 12 includes circuitry 30 for generating the pulses under the control of the microprocessor/controller 20.

The stimulation unit is also provided on its exterior surface with two electrical contacts 32, 34. These electrical contacts 32, 34 are positioned so that they match the positions of two spring electrical contacts 36, 38 on an exterior surface of the storage case 14.

The storage case 14 has a socket 40, for example a USB-C socket, allowing a mobile phone charger 42 to be used to connect a mains power supply to a battery charging controller 44. For example, the mobile phone charger 42 may provide a 5V supply to the battery charging controller 44. The battery charging controller 44 controls the charging of the battery 22 when the socket 40 is connected to the mains power supply, and when the electrical contacts 32, 34 are connected to the electrical contacts 36, 38.

In the embodiment illustrated in FIG. 1, the charging controller 44 is located in the stimulation unit 12. In other embodiments, the charging controller 44 is located in the storage case 14.

In other embodiments, the storage case 14 may be provided with a rechargeable battery that can be recharged whenever the socket 40 is connected to the mains power supply, allowing the battery 22 to be charged when the electrical contacts 32, 34 are connected to the electrical contacts 36, 38, even if the socket 40 is not connected to the mains power supply at that time.

In other embodiments, the storage case 14 and the stimulation unit 12 may be provided with wireless charging circuitry, for example using the Qi standard interface for wireless power transfer, allowing the battery 22 to be charged from the storage case 14, without requiring the electrical contacts 32, 34 to be connected to the electrical contacts 36, 38.

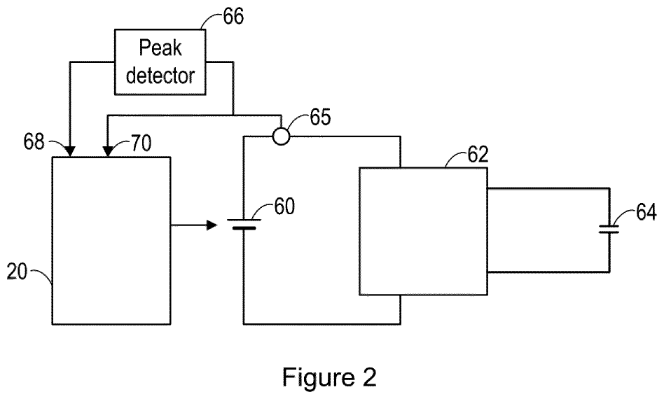

FIG. 2 shows in more detail the form of the electrical circuitry in the stimulation unit 12. Thus, the programmable controller 20 controls the voltage level provided by a voltage source 60, which is connected to pulse generation circuitry 62. The pulse generation circuitry 62 generates pulses that are applied to the electrodes 16, 18, which are shown in FIG. 2 as an effective capacitance 64. The pulse generation circuitry generates constant voltage pulses. That is, the magnitude of the voltage remains constant during one positive pulse and one negative pulse. However, the voltage may be varied for subsequent pulses, as described in more detail below, in order to take account of changing conditions.

In this example TENS device, the electrical bipolar pulse circuit generation circuitry 62 generates stimulation current pulses with specified characteristics. The pulse waveform specifications include intensity (mA), duration (μsec) and shape (typically monophasic or biphasic). The pulse pattern specifications include the frequency (Hz) of the stimulation pulses and the length of each continuous stimulation session (minutes). Just by way of illustrative example, the electrical stimulation is typically in the form of low intensity (typically less than 100 mA), short duration (typically 50-250 μsec) pulses at frequencies typically between about 10 and 200 Hz.

One significant safety concern for conventional TENS use, particularly when the user is sleeping, is the potential for “electrode peeling” (i.e., where the electrodes of the TENS device unintentionally separate from the skin of the user), resulting in an increased current and power density due to the decreased electrode-skin contact area. Thus, applying the same excitation to the skin over a reduced surface area may lead to a lack of comfort for the user, or even skin injuries.

As described in more detail below, in this device, the lack of contact between the electrodes 16, 18 and the user's skin surface is detected by considering the value of the current supplied to the electrodes during the pulses, for example the peak current values and the evolution of the current over time.

Thus, the current in the supply to the electrodes 64 is sensed by a current sensor 65, and a peak detector 66 detects a peak level in the current.

The programmable controller 20, which may for example be an ARM microprocessor such as Silicon Labs model EFR32MG1P132F256, in this embodiment has two A/D inputs 68, 70 for receiving analog signals from the electrical circuitry and converting them to digital signals for handling by the controller 20.

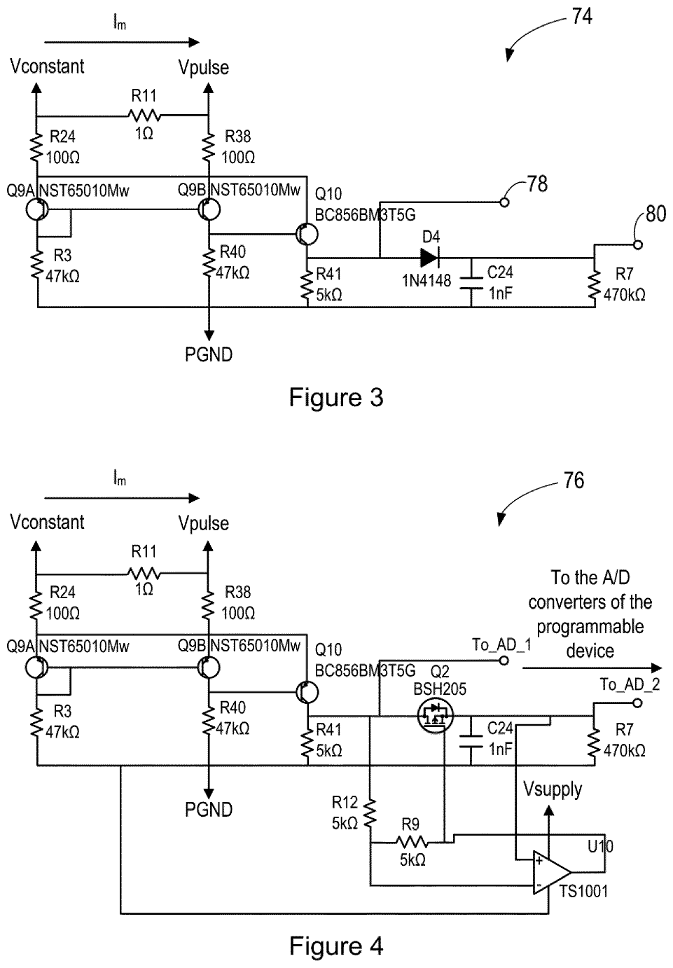

FIG. 3 shows in more detail the form of electrical circuitry 74, which incorporates the current sensor 65 and the peak detector 66.

In FIG. 3, Vconstant is the voltage provided by the constant voltage source 60 and Vpulse is the connection of the current sensor to the pulse generator circuit 62. The current to be measured, Im, flows through the resistor R11 and creates an imbalance at the emitters of the transistors Q9A and 09B. This decreases the voltage at the base of the transistor Q10 and increases its current yield until the imbalance is compensated. When this occurs, the voltage drop across the resistor R11 is mirrored across the resistor R24 by a current that is driven by Q10 so that the current to be measured, Im, is given by the following equation:

Im=R24*I10/R11,

where I10 is the current through Q10.

The output voltage at the output terminal 78 is the voltage across the resistor R41, and is given by:

R41*I10.

This voltage at the output terminal 78 can also be regarded as the voltage across R11 amplified by the ratio R41/R24. The voltage is measured by the analog to digital converter included in the controller 20.

The diode D4 and the capacitor C24 implement a detection of the maximum voltage at the output terminal 78, effectively implementing a peak detector of the voltage.

The design of the peak detector is such that R41 and C24 together have a time constant that is much less than the duration of the pulse. That is, in an example with a pulse duration of 250 μs:

R41*C24<<250 μs.

Equally, the design allows the discharge of the capacitor C24 through the resistor R7 after one pulse has ended, before a new pulse is produced. Thus, in an example with a pulse repetition rate of 200 Hz:

0.25 ms<R7*C24<5 ms.

Thus, the node 78 of the circuit shown in FIG. 3 is connected to the input terminal 70 of the controller 20, and the node 80 of the circuit shown in FIG. 3 is connected to the input terminal 68 of the controller 20.

FIG. 4 shows in more detail an alternative form of the electrical circuitry 76, where corresponding elements are indicated by the same reference numerals, but where the diode D4 in FIG. 2 has been substituted by a transistor Q2, an amplifier U10 and resistors R9 and R12. These three components work as an ideal diode effectively improving the detection of the maximum (peak) current at the node 80.

In the circuit of FIG. 4, the amplifier U10 makes the transistor Q2 conduct as long as the voltage across C24 is less than the voltage across the resistor R41. When the voltage across C24 becomes greater than the voltage across the resistor R41, the amplifier U10 switches off the transistor Q2, and the voltage across C24, which is actually the peak or maximum voltage across R41, is maintained.

As in FIG. 3, the node 78 of the circuit shown in FIG. 4 is connected to the input terminal 70 of the controller 20, and the node 80 of the circuit shown in FIG. 4 is are connected to the input terminal 68 of the controller 20.

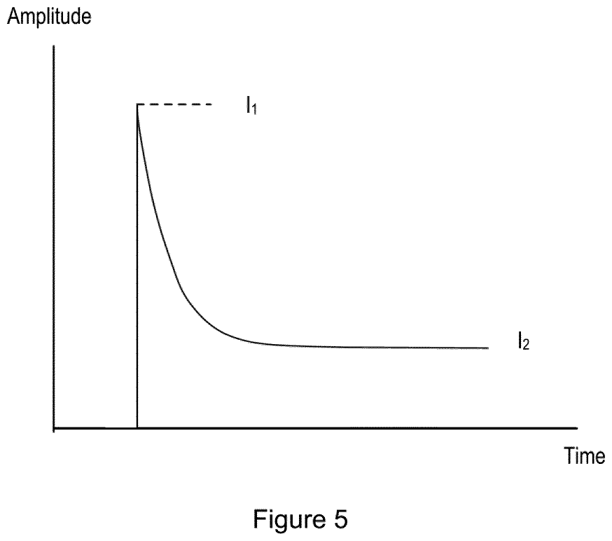

FIG. 5 shows the form of the current waveform that is obtained by the current measuring circuitry 74 or 76 upon the application of either a positive or negative voltage pulse over the patient's skin by the stimulating electrodes 16, 18.

In these embodiments, the programmable device 20 sets a constant voltage at the voltage source 60, with this constant voltage being selectable from within a range of 5 V to 50 V, for example, and the pulse generator circuit 62 excites the skin with a pulsed voltage, i.e. a voltage that is half of the time positive and half of the time negative (with a gap in between for electrical safety), with a pulse duration that may be between 50 μs and 250 μs at a variable frequency between 10 Hz and 200 Hz.

When the pulse of constant voltage Vpulse is applied to the user's skin, the current through the skin follows the form shown in FIG. 5, as is known from prior work.

At the start of the pulse, the current has a peak value, as a high current is needed to charge the skin capacitance across relatively low resistances (given by the addition of the electrode wiring resistance R_{electrode}, which can be measured during the manufacturing process and which does not change, and the resistance R₅ that represents the series resistance of the electrode itself). This peak current I₁ is given at the start of the pulse by:

$${I}_{1} = \frac{Vpulse}{{R}_{electrode} + {R}_{s}} \qquad ( 1 )$$

As the skin capacitance charges, the current decreases to a plateau value I₂. At this time, the current drawn from the voltage source also depends on the electrical resistance of the skin Rₚ, which is typically much bigger than the other resistances. Thus, I₂ is smaller than I₁. Specifically:

$${I}_{2} = \frac{Vpulse}{{R}_{electrode} + {R}_{s} + {R}_{p}} \qquad ( 2 )$$

As shown in the document A. van Boxtel, “Skin resistance during square-wave electrical pulses 1 to 10 mA”, Med. & Biol. Eng & Comput., 1977, 15, 679-687, the continuous usage of a skin electro stimulation device decreases the value of Rₚ and hence the value of 12 typically increases over the duration of the treatment, with the value of 11 being unaffected.

Then, if one of the electrodes 16, 18 starts to peel off the user's skin, leading potentially to the risk of an increased current density being applied to the user's skin if not corrected, the electrode resistance Rₛ will increase. This will be detectable directly in changes over time to the value of I₁, whereas its effect on I₂ may be masked (and even counteracted) by the expected decrease in the value of Rₚ.

Thus, the strategy here is to detect the peel-off of the electrode by measuring the values of the currents I₁ and I₂ during each stimulation pulse, using the analog to digital converter of the digital programmable device 20, fed by the circuits 74 or 76, and following its evolution over time.

In normal working conditions the system will detect a constant value of I₁ and a slight decrease in the value of I₂. However, the detection of a decrease in I₁ and I₂ simultaneously at a constant level of stimulation implies the peel-off of the electrode.

Initially, it is assumed that the electrode is correctly attached to the user's skin. This allows the controller 20 to calculate an initial value of the resistances Rₛ and Rₚ by measuring I₁ and I₂, using the above equations (1) and (2), and assuming that the value of R_{electrode} was measured at the manufacture of the electrode. In addition, the initial value of Vconstant is stored. If the electrode starts to peel off the user's skin, the resistances Rₚ and Rₛ increase, and hence the values of I₁ and I₂ both decrease. If this is detected, the voltage Vpulse is decreased. Because the resistor R11 has a low resistance, the voltage Vpulse is very similar to the voltage Vconstant, and so Vpulse is decreased by decreasing the reference voltage of the power supply that generates Vconstant, and hence decreases the value of Vconstant. Reducing Vpulse reduces the current, and hence maintains the power density applied through the user's skin substantially constant. Moreover, following equations (1) and (2) above, the ratio of I₂ to I₁ is given by I₂/I₁=(R_{electrode}+Rₛ+Rₚ)/(R_{electrode}+Rₛ), where R_{electrode} is typically of the order of tens of ohms, Rₛ is typically of the order of hundreds of ohms, and Rₚ is typically of the order of kiloohms. The ratio of I₂ to I₁ remains substantially unchanged as the voltage is reduced. This reduction is continued until it is determined that the value of the resistance (Rₛ+Rₚ) has increased by a predetermined amount (for example, doubled) from its initial value, which is detected by a decrease in the voltage 60 (Vconstant) to a predetermined level (which may for example be 75%, or 50%, or 25% of its original stored value). At this point, only a fraction (for example only 50%) of the electrode surface is still attached, which is considered a safe use.

Thus, the peak detector circuit as shown in FIG. 3 or FIG. 4 is used in order to make detection easier and to avoid the need for a very fast sampling of the value of I₁.

The device is also able to detect an improper electrical connection or no connection of the electrodes to the skin. This occurs if I₁=I₂ (or equal up to 90%) and is produced by very big Rₛ voltage values and no current injection by the driving circuit 62 due to the absence of skin resistance after charging the capacitance of the electrode.

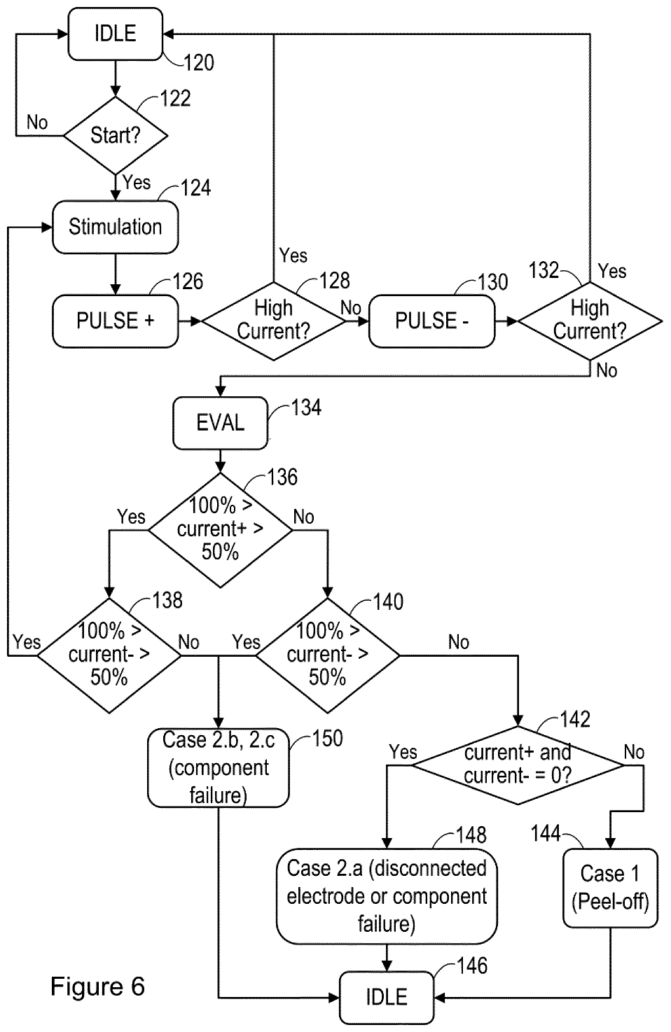

FIG. 6 is a flow chart, illustrating the process for fault detection.

At step 120, the process is idle and, at step 122, it is determined that the process should start. The stimulation then begins in step 124. In step 126, a positive voltage pulse is generated, and it is determined at step 128 whether the current value (i.e. the value of the peak current I1 as shown in FIG. 5) is excessive. If the current value is normal, in step 130, a negative voltage pulse is generated, and it is determined at step 132 whether the current value is excessive.

Assuming that the current values are not excessive, the process passes to step 134, where the current values, current+ and current−, from the positive and negative voltage pulses respectively, are evaluated by the circuit of FIG. 3 or FIG. 4.

In step 136, it is determined whether the current from the positive pulse, current+, is within the range from 50% to 100% of the nominal value. It is then determined, either in step 138 or step 140, whether the current from the negative pulse, current-, is within the range from 50% to 100% of the nominal value. The nominal values of the current are taken from an initial measurement, when it is assumed that the electrodes are correctly attached to the user's skin.

If it is determined in step 136 and step 138 that the currents from the positive pulse and from the negative pulse are both within the range from 50% to 100% of the nominal value, the process returns to step 124 and a new cycle begins.

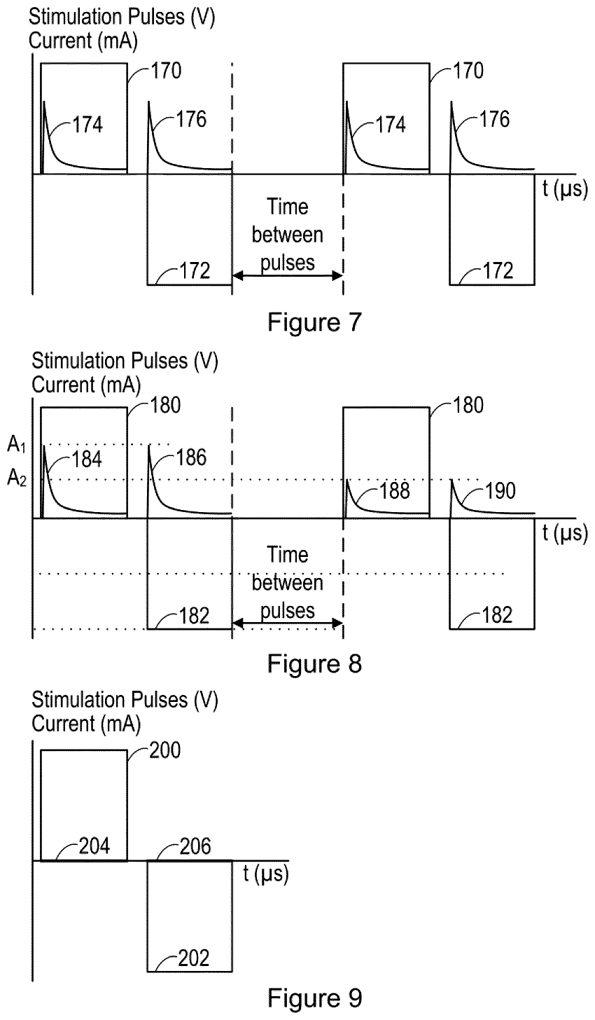

FIG. 7 shows this situation of normal operation, where positive voltage pulses 170 and negative voltage pulses 172 both produce the expected current pulses 174, 176.

However, in the event that there is no longer a proper contact of the surface electrodes, because the electrode surface has started to peel off the user's skin, as mentioned above, there is a risk of an excessive stimulation current or power density, with the possibility of pain to the user and even thermal burns in extreme cases. Providing stimulation by voltage pulses (rather than by current pulses) provides some inherent protection in this situation, because the stimulation current decreases as the electrode peels off (rather than being maintained if constant current pulses are used).

In FIG. 6, if it is determined in step 136 and step 140 that the currents from the positive pulse and from the negative pulse are both outside the range from 50% to 100% of the nominal value, the process passes to step 142. In step 142, it is determined whether the currents are equal to zero.

If the currents are non-zero, it is assumed at step 144 that the electrode is peeling off. This is illustrated in FIG. 8 shows a situation where positive voltage pulses 180 and negative voltage pulses 182 initially produce the current pulses 184, 186 of the expected amplitude A₁, but subsequently produce the current pulses 188, 190 of a reduced amplitude A₂. The ratio between A₂ and A₁ is determined by the increase in Rs during the peel-off, and by the regulation of the value of Vconstant; as mentioned above the stimulator switches off if A₂ is 25% of A₁.

As mentioned above, the intention is that, when the electrode starts to peel off, the applied voltage is decreased (and as a consequence the current also decreases) to keep the power density constant until it is determined that the skin resistance has reached 200% of the initial value, which translates to 50% of the electrode surface still being attached, which is considered safe.

If this condition is met, the device is automatically switched off at step 146.

The method shown in FIG. 6 also identifies various possible failure mechanisms.

Thus, if it is determined at step 142 that the currents are equal to zero, this may mean that no current is flowing between the electrodes, for example due to a malfunction of the supply voltage or the voltage generation circuit or because the electrode is not connected. This is shown in FIG. 9, where positive voltage pulses 200 and negative voltage pulses 202 both produce zero currents 204, 206. In this case, the process passes to step 148, and then the device is automatically switched off at step 146.

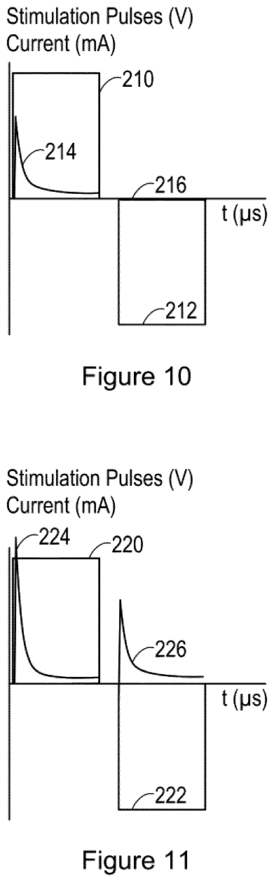

FIG. 10 shows a further possibility, where positive voltage pulses 210 produce current pulses 214, and negative voltage pulses 212 produce zero currents 216. (Or, equivalently, positive voltage pulses produce zero currents, and negative voltage pulses produce current pulses.) If one of these pulses drops to zero, while the other one is still measuring the expected value, this may mean that one of the sides of the electrodes driving circuitry is failed to open circuit.

This is detected at step 150 of the process shown in FIG. 6, if it is determined in step 136 and step 138, or in step 136 and step 140, that one of the currents from the positive pulse and from the negative pulse are inside the range from 50% to 100% of the nominal value, and one of the currents is outside that range. Again, in this case, the process passes to step 146, and the device is automatically switched off.

Another possible error condition, in where one of the transistors in the circuitry fails to short circuit, which will result in a higher current in one of the bipolar pulses than expected.

This is illustrated, as an example, in FIG. 11, in which positive voltage pulses 220 produce abnormal current pulses 224, while negative voltage pulses 222 produce the expected current pulses 226.

This is detected in step 128 or step 132 of FIG. 6. If this condition is detected, the device is switched off.

A feature of the device shown in FIG. 1 is that it is provided with an integrated accelerometer 28 for controlling operational functions. This means that the device does not need to be provided with buttons, and the user can communicate with the device by tapping on the device or in the near vicinity of the device. This works even if the device is placed under clothing, which makes it possible to operate the stimulation unit 12 easily and without anyone noticing it while in public, or while lying in bed, without the difficulty of finding a button in the dark.

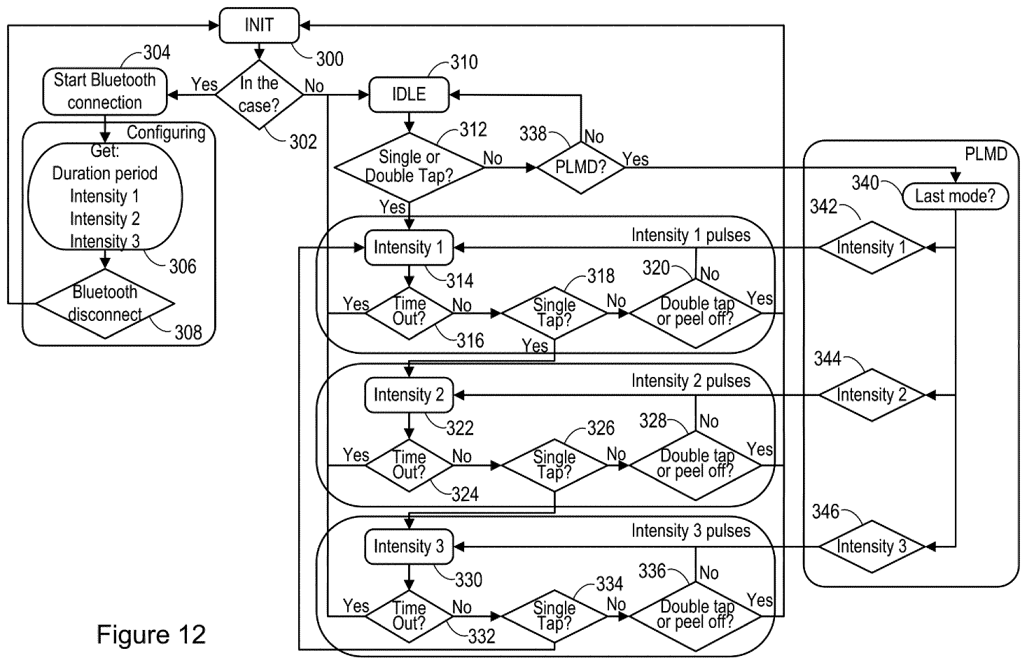

FIG. 12 is a flow chart, illustrating a method of control of the device. This method is implemented in software running on the microprocessor 20 in the stimulation unit.

The method is initialised at step 300, and at step 302 it is determined whether the stimulation unit 12 is in the storage case 14. This can be determined by detecting whether the electrical contacts 32, 34 are in contact with the charging pins 36, 38.

If it is determined that the stimulation unit 12 is in the storage case 14, the process passes to step 304, in which the microprocessor 20 initiates a Bluetooth™ connection between the communications module 24 and any suitable processing device such as a smartphone or tablet. In this example, the connection is with the computer 26. This can allow the device to be configured, either by a medical professional running suitable software, or by the user themselves by means of a suitable app.

By way of example, step 306 shows configuration of the device including setting a pulse duration period, and three intensity levels, identified here as Intensity 1, Intensity 2, and Intensity 3. Each of these intensity levels corresponds to a particular pulse voltage, and these can be chosen to give the desired intensity levels, based on equations (1) and (2) above.

After the configuration is complete, the Bluetooth™ link may be disconnected, and the process returns to step 300.

If it is determined at step 302 that the stimulation unit 12 is not in the storage case 14, but instead is attached to the body by the electrode, the process passes to step 310, in which the device is initially idle.

In this embodiment, the operation of the device can be controlled by means of the user tapping on or near the device, such that the taps can be detected by the accelerometer 28. In this example, two predetermined patterns of user-generated taps are defined.

Specifically, by way of example, the first predetermined pattern of user-generated taps may be a single tap, that is, a distinct tap within a predetermined characteristic, while the second predetermined pattern of user-generated taps may be a double tap, that is, two distinct taps with predetermined characteristics within a predetermined time.

As described in more detail below, an intensity of the pulses is altered in accordance with a predetermined sequence of intensities in response to the first predetermined pattern of user-generated taps. In this example, the predetermined sequence of intensities is: a lowest intensity, an intermediate intensity, and a highest intensity, followed by a return to the lowest intensity. As also described in more detail below, the device is deactivated in response to the second predetermined pattern of user-generated taps.

Thus, in step 312, it is determined whether a single or double tap is detected.

If a single or double tap is detected in step 312, the process passes to step 314, in which the intensity is set to the lowest intensity level, Intensity 1, that was set during the configuration in step 306.

Stimulation pulses are then generated and applied to the user. A timer is started and, if a maximum activation time (for example in the range of 15 to 30 minutes) is reached before there is any further control activity, it is determined in step 316 that a time out threshold has been reached, and the device is deactivated and returns to the idle state in step 310.

If the time out threshold has not been reached, it is determined in step 318 whether a single tap has been detected. If not, it is determined in step 320 whether a double tap has been detected or whether the process of FIG. 6 identifies that an electrode has peeled off to an extent that the stimulation should be stopped. If so, the device is switched off, and the process returns to step 300. If it is determined in step 320 that a double tap has not been detected, the process returns to step 314, and the pulse intensity is maintained at Intensity 1.

If it is determined in step 318 that a single tap has been detected, the process passes to step 322, in which the intensity is set to the intermediate intensity level, Intensity 2, that was set during the configuration in step 306.

Stimulation pulses are then generated and applied to the user. If the maximum activation time is reached before there is any further control activity, it is determined in step 324 that a time out threshold has been reached, and the device is deactivated and returns to the idle state in step 310.

If the time out threshold has not been reached, it is determined in step 326 whether a single tap has been detected. If not, it is determined in step 328 whether a double tap has been detected or whether the process of FIG. 6 identifies that an electrode has peeled off to an extent that the stimulation should be stopped. If so, the device is switched off, and the process returns to step 300. If it is determined in step 328 that a double tap has not been detected, the process returns to step 322, and the pulse intensity is maintained at Intensity 2.

If it is determined in step 326 that a single tap has been detected, the process passes to step 330, in which the intensity is set to the highest intensity level, Intensity 3, that was set during the configuration in step 306.

Stimulation pulses are then generated and applied to the user. If the maximum activation time is reached before there is any further control activity, it is determined in step 332 that a time out threshold has been reached, and the device is deactivated and returns to the idle state in step 310.

If the time out threshold has not been reached, it is determined in step 334 whether a single tap has been detected. If not, it is determined in step 336 whether a double tap has been detected or whether the process of FIG. 6 identifies that an electrode has peeled off to an extent that the stimulation should be stopped. If so, the device is switched off, and the process returns to step 300. If it is determined in step 336 that a double tap has not been detected, the process returns to step 330, and the pulse intensity is maintained at Intensity 3.

If it is determined in step 334 that a single tap has been detected, the process returns to step 314, in which the intensity is set back to the lowest intensity level, Intensity 1.

In addition, if at step 312 a single or double tap is not detected, the process passes to step 338, in which it is determined whether the accelerometer 28 detects a pattern of movement that is characteristic of the symptoms of periodic limb movement disorder (PLMD). If so, the device is activated automatically. Specifically, in this embodiment, the process passes to step 340, in which it determines the last used mode (i.e. the intensity level when the device was last deactivated). Thus, it is determined in steps 342, 344, and 346 whether the device was last used at the lowest intensity, the intermediate intensity, or the highest intensity, and the process passes to step 314, 322, or 330, as appropriate.

In other embodiments, when the accelerometer 28 detects a pattern of movement that is characteristic of the symptoms of periodic limb movement disorder (PLMD), the process may simply pass to step 314 (or, in other embodiments, to step 322 or 330), without identifying the intensity level when the device was last deactivated.

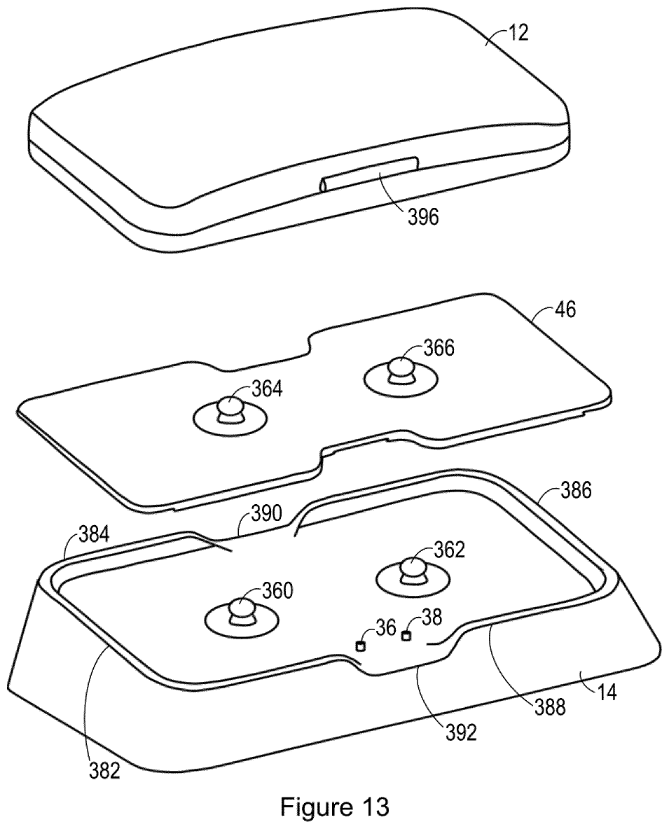

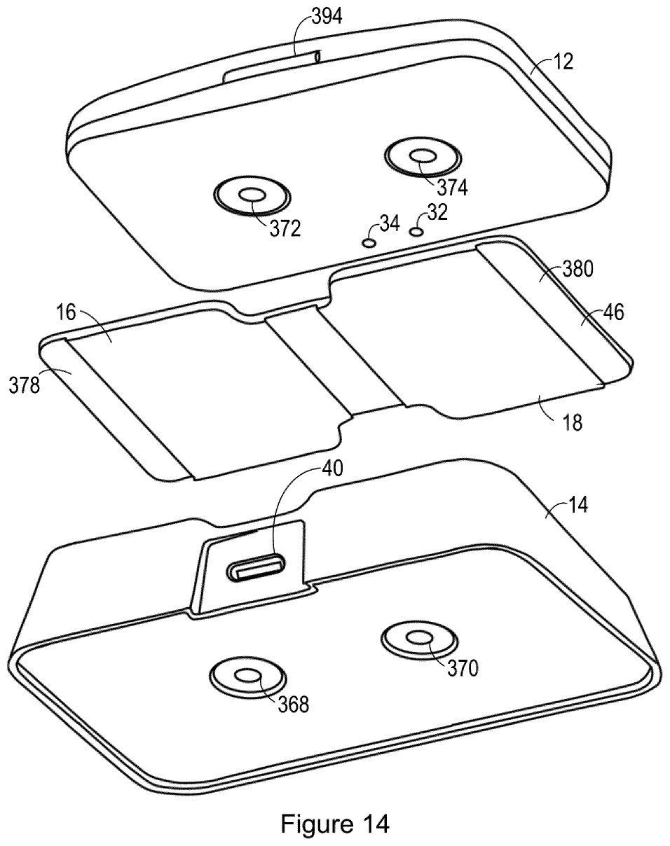

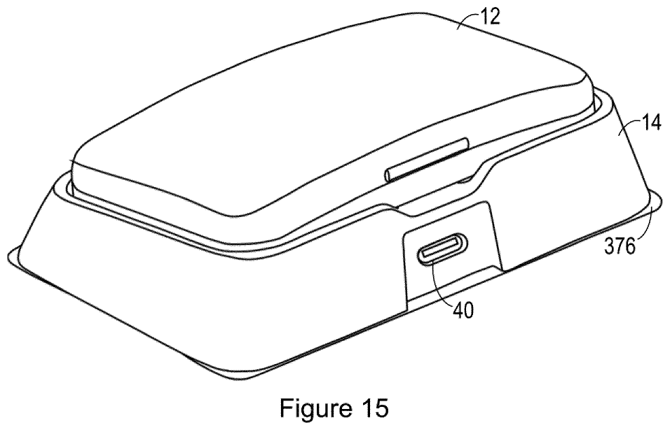

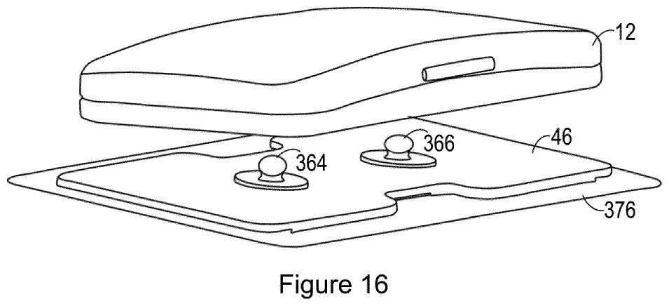

FIG. 13, FIG. 14, FIG. 15, and FIG. 16 show the structure of the stimulation unit 12 and storage case 14. Specifically, FIG. 13 shows a view from above and from the front of the stimulation unit 12 separated from the storage case 14, also showing an electrode patch 46; FIG. 14 shows a view from below and from the rear of the stimulation unit 12 separated from the storage case 14, again also showing the electrode patch 46; FIG. 15 shows a view from above of the stimulation unit 12 inside the storage case 14; and FIG. 16 shows a view from above of the stimulation unit 12 and the electrode patch 46.

The upper surface of the storage case 14 has a pair of protrusions 360, 362, which are made of magnetic material. Similarly, the upper surface of the electrode patch 46 has a corresponding pair of protrusions 364, 366, which are made of magnetic material.

The lower surface of the storage case 14 has a pair of recesses 368, 370, which are surrounded by magnetic material. Similarly, the lower surface of the stimulation unit 12 has a corresponding pair of recesses 372, 374, which are surrounded by magnetic material.

Thus, when the device is charging, or being stored, the stimulation unit 12 can be placed in the storage case 14, as shown in FIG. 15, with the protrusions 360, 362 held in place in the recesses 372, 374 by the magnetic force. At the same time, the electrode patch 46 can be stored underneath the storage case 14, with the protrusions 364, 366 held in place in the recesses 368, 370 by the magnetic force. FIG. 15 shows a protective cover 376 on the lower surface of the electrode patch 46.

When the device is in use, the electrode patch 46 can be connected to the lower surface of the stimulation unit 12, with the protrusions 364, 366 held in place in the recesses 372, 374 by the magnetic force. FIG. 16 shows this arrangement, with the electrode patch 46 slightly separated from the lower surface of the stimulation unit 12 for clarity. FIG. 16 also shows the protective cover 376 on the lower surface of the electrode patch 46. The protective cover can be kept in place until it is desired to attach the electrodes to the user's skin, and can then be removed.

When the device is in use, the current therefore flows from the stimulation unit 12, through the magnetic material in and surrounding the recesses 372, 374, then through the magnetic material in and surrounding the protrusions 364, 366 on the electrode patch 46, and then to the electrodes 16, 18. From there, the current flows through the hydrogel material on the electrodes 16, 18 to the user.

As described above, this portable design means that the storage case 14 which protects the stimulation unit 12 also acts to provide a charging point for the stimulation unit 12. The storage case allows the device to be kept safe and discretely in a pocket and/or handbag. Furthermore, the design of the stimulation unit 12 makes the device easy to use in public, without being visible to other people. The stimulation unit 12 can be placed on the skin, unseen under clothing, while the storage case 14 can be put back discretely into the pocket or handbag. With the proposed configuration of the storage case it is not possible to charge the stimulator when it is placed on the skin, therewith making the device inherently safe.

FIG. 13 shows the electrical contacts 36, 38 on the storage case 14, and FIG. 14 shows the electrical contacts 32, 34 on the stimulation unit 12. The electrical contacts 36, 38 are spring contacts, guaranteeing a good electrical contact between the pairs of electrical contacts when the stimulation unit 12 is held in the storage case 14.

FIG. 14 shows the electrodes 16, 18 on the lower surface of the electrode patch 46. FIG. 14 also shows non-conductive regions 378, 380 at the ends of the electrode patch 46, allowing the electrode patch to be handled (for example when placing it on the user's skin) without needing to touch the electrodes 16, 18. The arrangement whereby the electrodes 16, 18 contact the stimulation unit 12 in the same way as the storage case 14 contacts the stimulation until means that it is not possible for a patient to wear the device (with the electrodes in contact with the stimulation unit) while the stimulation unit is recharging. The stimulation unit 12 must be placed in the storage case 14 to be recharged, and it is not possible at the same time to place the electrodes 16, 18 in contact with the patent's skin.

The side and end walls 382, 384, 386, 388 of the storage case 14 extend above the upper surface that has the protrusions 360, 362. Thus, as shown in FIG. 15, the stimulation unit 12 is held in place within the storage case 14 by a) a magnetic force between the protrusions 360, 362 and the recesses 372, 374, and b) the walls 382, 384, 386, 390 of the storage case. The longer side walls 384, 388 have respective notches 390, 392 in them, so that the stimulation unit 12 can easily be picked out of the storage case 14 by the user even when it is held in place, using grips 394, 396 on the sides of the stimulation unit 12.

FIGS. 14 and 16 also show the location of the socket 40, for example a USB-C socket, on the storage case 14.

There is thus disclosed a Transcutaneous Electrical Nerve Stimulation, TENS, device, and methods of operation thereof, that provide convenient and effective operation.

1. A method of operation of a Transcutaneous Electrical Nerve Stimulation, TENS, device, the method comprising:
while the device is in operation, generating pulses to be applied to a pair of electrodes;
using an accelerometer to detect taps on the device;
in response to a first predetermined pattern of user-generated taps, altering an intensity of said pulses in accordance with a predetermined sequence of intensities; and
in response to a second predetermined pattern of user-generated taps, deactivating the device.

2. A method according to claim 1, wherein the first predetermined pattern of user-generated taps comprises one tap within a first predetermined time period.

3. A method according to claim 1, wherein the second predetermined pattern of user-generated taps comprises two taps within a second predetermined time period.

4. A method according to claim 1, wherein the predetermined sequence of intensities comprises a series of increasing intensities followed by a return to a lowest intensity.

5. A method according to claim 4, wherein the predetermined sequence of intensities comprises a series of three increasing intensities followed by a return to the lowest intensity.

6. A Transcutaneous Electrical Nerve Stimulation, TENS, device, the device comprising:
pulse generation circuitry, for generating pulses to be applied to a pair of electrodes;
an accelerometer, for detecting taps on the device; and
a processor, wherein the processor is programmed to:
while the device is in operation, use the pulse generation circuitry to generate pulses to be applied to the pair of electrodes;
use the accelerometer to detect taps on the device;
in response to a first predetermined pattern of user-generated taps, alter an intensity of said pulses in accordance with a predetermined sequence of intensities; and
in response to a second predetermined pattern of user-generated taps, deactivate the device.

7. A method of operation of a Transcutaneous Electrical Nerve Stimulation, TENS, device, the method comprising:
applying constant voltage pulses to a pair of electrodes attached to the skin of a user;
during a plurality of said pulses, detecting a peak current at the start of the pulse, and detecting a plateau current at the end of the pulse;
comparing peak currents from the plurality of pulses;
comparing plateau pulses from the plurality of pulses; and
determining that at least one of the electrodes is peeling if the peak currents from the plurality of pulses decline over time and if the plateau pulses from the plurality of pulses decline over time.

8. A method according to claim 7, further comprising:
detecting a pulse characteristic indicating a failure of a component in pulse generating circuitry of the TENS device.

9. A Transcutaneous Electrical Nerve Stimulation, TENS, device, the device comprising:
a pair of electrodes, for attachment to the skin of a user; and
a processor, wherein the processor is programmed to:
apply constant voltage pulses to the pair of electrodes attached to the skin of the user;
during a plurality of said pulses, detect a peak current at the start of the pulse, and detect a plateau current at the end of the pulse;
compare peak currents from the plurality of pulses;
compare plateau pulses from the plurality of pulses; and
determine that at least one of the electrodes is peeling if the peak currents from the plurality of pulses decline over time and if the plateau pulses from the plurality of pulses decline over time.

10-23. (canceled)

24. A TENS device according to claim 6, wherein the first predetermined pattern of user-generated taps comprises one tap within a first predetermined time period.

25. A TENS device according to claim 6, wherein the second predetermined pattern of user-generated taps comprises two taps within a second predetermined time period.

26. A TENS device according to claim 6, wherein the predetermined sequence of intensities comprises a series of increasing intensities followed by a return to a lowest intensity.

27. A TENS device according to claim 26, wherein the predetermined sequence of intensities comprises a series of three increasing intensities followed by a return to the lowest intensity.

28. A TENS device according to claim 9, wherein the processor is programmed to detect a pulse characteristic indicating a failure of a component in pulse generating circuitry of the TENS device.
